(19) United States
(12) Patent Application Publication
(10) Pub. No.: US 2025/0027173 A1
(43) Pub. Date: Jan. 23, 2025

(54) DNA CLOCKS CAPABLE OF LONG-TERM TIMEKEEPING COMPRISING A COMPOSITION FOR MEASURING OR PREDICTING THE DURATION OF CELLULAR EVENTS IN CELLS

(71) Applicant: INDUSTRY-ACADEMIC COOPERATION FOUNDATION, YONSEI UNIVERSITY, Seoul (KR)

(72) Inventors: Hyongbum KIM, Seoul (KR); Ji-Hye PARK, Seoul (KR); Hanahrae LEE, Seoul (KR)

(21) Appl. No.: 18/741,498

(22) Filed: Jun. 12, 2024

(30) Foreign Application Priority Data
Jun. 13, 2023 (KR) 10-2023-0075529

(51) Int. Cl. C12Q 1/6897; C12Q 1/6811; C12Q 1/6874

(52) U.S. Cl. CPC C12Q 1/6897; C12Q 1/6811; C12Q 1/6874

(57) ABSTRACT

Disclosed are a DNA clock capable of recording long periods of time, comprising a composition for measuring or estimating elapsed time in cells, and a method of measuring or estimating elapsed time in cells using the composition.

### CROSS-REFERENCE TO RELATED APPLICATION

The priority of Korean Patent Application 10-2023-0075529 filed Jun. 13, 2023 is hereby claimed under the provisions of 35 USC § 119, and the disclosure thereof is hereby incorporated herein by reference in its entirety, for all purposes.

### SEQUENCE LISTING

This application includes an electronically submitted sequence listing in .xml format. The .xml file contains a sequence listing entitled “727 CorrectedSeqListing.xml” created on Aug. 26, 2024 and is 14,584 bytes in size. The sequence listing contained in this .xml file is part of the specification and is hereby incorporated by reference herein in its entirety.

### BACKGROUND OF THE INVENTION

### Field of the Invention

The present invention relates to a DNA clock capable of recording long periods of time, comprising a composition for measuring or estimating elapsed time in cells, and a method of measuring or estimating elapsed time in cells using the composition.

### Description of the Related Art

Chromosomal DNA provides an excellent means for writing biological information as well as storing the same in appropriate “memory” devices.

This DNA is not only structurally durable, but also has advantages such as compatibility and cost-effectiveness. DNA is an excellent storage medium, and its storage capacity can be amplified by a variety of molecular biology tools. With advances in biological systems, it is extremely important to keep track of multiple simultaneously occurring biological activities. DNA writers are genetic devices that have dual functions: writing the code of life as well as enabling to bring about modifications in the living cells through mechanisms like base substitutions, deletions, inversions and insertions.

Biological life is one of the most complex and dynamic systems in nature. Through evolution and natural selection, vast biochemical and biological diversity has emerged, from complex molecules to multicellular life. These multi-scale biological systems precisely generate and respond to a myriad of biotic signals of varying order and magnitude. Signals can take the form of ions, metabolites, nucleic acids or proteins, producing biochemical gradients and signaling cascades that propagate across many length and time scales within cells and across populations. The integration of these signals through genetic and epigenetic regulation at the transcriptional, translational and post-translational levels results in robust cellular behaviors.

The diverse and enormous number of signals inducing changes in the cell is extremely difficult to be kept track of. With the advent of genomics, DNA can be used as an excellent writing means as well as a storage medium for overcoming the stereotypic difficulties associated with traditional methods of storing biological information (Science. 2018 Aug. 31; 361(6405): 870-875).

DNA is the fundamental molecule by which information is stored and utilized to produce life. DNA is a high-density storage medium that can be quickly copied by exponential polymerase chain reaction (PCR) amplification and stably preserved. Biological information encoded in DNA can be directly converted into actionable cellular responses through gene regulation and expression.

Many molecular events that occur in biological systems are transient and thus difficult to monitor and study within their native context. However, DNA writing can be used to create molecular recorders that capture these transient signals and stably encode them into the DNA of cell populations or individual cells. Although gene regulation and gene expression mechanisms can be utilized to convert biological activities into cellular signals, these signals have to be stored in a specific format. Nucleic acid sequencing and Next Generation Sequencing (NGS) have harnessed several components of cell lifecycle events like adaptive immunity, phase variation systems, arrangements in the genome, and retron-mediated recording systems. Thereamong, one of the most popular molecular recording devices is the CRISPR Cas-based molecular recording device.

The CRISPR-Cas-based base editor has a limitation in that, since the function thereof is limited to substituting specific bases, it is impossible to write various base sequence patterns to predefined targets, making multiplex recording difficult. However, the present inventors have engineered a previously reported ‘DNA Typewriter’ system to a long-term time recording system (“long-term DNA clock”), which is even capable of multiplex recording in future, using the CRISPR-Cas-based prime editor and the ‘DNA Tape’, which is a target sequence having repeating sequences capable of recording various edit patterns (Choi J et al., Nature (2022)). The present inventors have developed a DNA tape construct having monomers that are repeating units, each containing a disrupted protospacer adjacent motif (PAM), and have found that the DNA tape construct may be used to record long periods of time, thereby completing the present invention.

### SUMMARY OF THE INVENTION

An object of the present invention is to provide a composition for measuring or estimating elapsed time in isolated cells.

Another object of the present invention is to provide a method for measuring or estimating elapsed time in cells using the composition.

To achieve the above objects, the present invention provides a composition for measuring or estimating elapsed time in isolated cells, comprising:
(a) a prime editor protein or a nucleic acid encoding the same;
(b) a DNA tape comprising a disrupted monomer, which includes a disrupted protospacer adjacent motif (PAM), and active monomers, each including a PAM; and
(c) a prime editing guide RNA (pegRNA) which comprises a spacer and a reverse transcription template (RTT) and recognizes the DNA tape.

The present invention also provides isolated cells having introduced therein:
(a) a nucleic acid that encodes a prime editor so that the prime editor is expressed or is planned to be expressed;
(b) a DNA tape comprising a disrupted monomer, which includes a disrupted PAM, and active monomers, each including a PAM; and
(c) a prime editing guide RNA (pegRNA) which comprises a spacer and a reverse transcription template (RTT) and recognizes the DNA tape.

The present invention also provides a method for measuring or estimating elapsed time in isolated cells, comprising steps of:
transducing the composition into the isolated cells and then culturing the cells;
harvesting the cultured cells at any time point t, and then analyzing whether a sequence has been inserted into the DNA tape from the genomic DNA of the cells;
based on analyzing whether the sequence has been inserted into the DNA tape, measuring the fraction of an inserted sequence at each insertion site in the copies of the DNA tape sequence at time t, that is, an insertion frequency (IF_{i,t}) at i^{th} insertion site at time t;
measuring the frequency of the copy number of an intact sequence at each insertion site in the total copy number of the DNA tape sequence at time t, that is, the frequency of intact sequence (F_{i,t}) at i^{th} insertion site at time t; and
calculating the time elapsed from a given time point using the following equation:

$${F}_{i , t} = 1 - {IF}_{i , t} = {e}^{- {λ}_{i} ( t - {t}_{0} )} ( t \geq 0 , {t}_{0} \geq 0 )$$

wherein F_{i,t} represents the frequency (fraction) of the copy number of an intact sequence at the i^{th} insertion site relative to the total copy number of the DNA tape, analyzed at any time point t, IF_{i,t} represents the fraction of an inserted sequence at the i^{th} insertion site, measured at any time point t, λᵢ is a positive constant that represents the rate of sequence insertion by prime editing at the i^{th} insertion site per unit time, and t₀ is the latent time taken for the composition transduced into the cells to be expressed.

### BRIEF DESCRIPTION OF THE DRAWINGS

FIG. 1 shows a vector construct used to produce PE2max knock-in cells and the expression of mRFP fluorescence by the produced PE2max knock-in cells.

FIG. 2 schematically shows a lentiviral vector constructed by cloning a DNA tape, which comprises a target sequence, and a prime editing guide RNA that can cause 3-bp sequence insertion and 1-bp substitution simultaneously in the DNA tape.

FIG. 3 schematically shows the structure of a DNA tape used in a novel DNA Typewriter system of the present invention and the structure of the DNA tape after repeated sequence insertions.

FIG. 4 schematically shows the working of the novel DNA Typewriter system of the present invention based on the sequence used in the examples, with the following sequences being shown in FIG. 4: nnnnnntcgnnnnnnnnnnntggnnnnnnnnnnntggnnnnnnnnnnntggnnnnnnnnnnn (SEQ ID NO: 6); nnnnnntcgnnnnnnnnnnnnnntcgnnnnnnnnnnntggnnnnnnnnnnntggnnnnnnnn nnn (SEQ ID NO: 7); and nnnnnntcgnnnnnnnnnnnnnntcgnnnnnnnnnnnnnntcgnnnnnnnnnnntggnnnnn nnnnnn (SEQ ID NO: 8).

FIG. 5 is a graph showing the insertion fraction (%) depending on the number of sequences inserted into the DNA tape sequence at each measurement time point.

FIG. 6 shows the results of comparing the true elapsed time with the elapsed time estimated based on an exponential decay curve fitted to experimental data.

FIGS. 7A and 7B show the monomer deletions (FIG. 7A), estimated elapsed time, and mean RAE values (FIG. 7B) that occurred as a result of an experiment conducted using the TAPE-1 sequence provided in the DNA Typewriter paper.

FIGS. 8A and 8B show the monomer deletions (FIG. 8A), estimated elapsed time, and mean RAE values (FIG. 8B) that occurred as a result of an experiment conducted using a DNA Typewriter sequence predicted to have highest prime editing efficiency according to the DeepPrime algorithm, among all possible DNA Typewriter constructs.

FIG. 9 shows the change in number of monomers for 28 days when using the engineered DNA Typewriter construct (using the sequence described in Example 2) according to the present invention, and indicates that the relative proportion of reads with deleted monomers do not tend to increase compared to when using a conventional DNA Typewriter construct.

### DETAILED DESCRIPTION OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

DNA is well suited to serve as a digital medium for in vivo molecular recording. However, DNA-based memory devices are constrained in terms of the number of distinct ‘symbols’ that can be concurrently recorded or by failure to capture the order in which events occur. DNA Typewriter, a general system for in vivo molecular recording that overcomes these and other limitations, was proposed (Choi J et al., Nature (2022)).

For a DNA Typewriter, ‘DNA tape’ is used as a recording medium. DNA tape is a sequence consisting of repeats of a 14-bp monomer. In this system, the prime editing guide RNA mediates the short insertional edits between monomers while shifting the position of the ‘write-head’ by one unit along the DNA tape, and involves successive genome editing.

Specifically, the DNA Typewriter includes a structure in which the editing site shifts forward by one monomer after sequence insertion occurs in the editing site, which acts as a write-head, in each editing round.

At this time, the 20-bp spacer of the prime editing guide RNA of the DNA Typewriter binds to the appropriate 20-bp write-head sequence and works, while the 17-bp length thereof may also bind to other monomers that must act in subsequent rounds rather than the write-head sequence in the corresponding editing round, and sequence insertion may occur at a site other than the editing site for the corresponding round. In this case, there is no guarantee that sequence insertion occurs sequentially while the write-head shifts forward by one monomer.

Accordingly, the present inventors have developed a novel DNA Typewriter system. This DNA Typewriter system is a system designed by reducing the sequence length, at which the guide RNA can bind to monomers in the 3′ direction with respect to the editing site, to 12 bp (less than 16 bp) so that the guide RNA cannot edit sites other than the editing site for the corresponding round.

According to the present invention, to overcome the monomer deletion-related problem occurring in the conventional DNA Typewriter system, in the DNA tape sequence with repeated monomers, 1) the length at which the spacer can bind to the remaining portions other than the target for the corresponding round in the DNA tape sequence is set to be shorter than 16 bp, and 2) the PAM in the monomer sequence where prime editing occurred, allowing successive sequence insertions to proceed in the 5′→3′ direction in the DNA tape sequence.

It is known that the minimum length, at which the spacer of the guide RNA spacer can bind to the target sequence and induce nicking with nuclease, is 16 bp (Dahlman J E et al., Nature Biotechnology (2015)). Therefore, the system of the present invention is a system that minimizes the possibility of occurrence of monomer deletions, observed in the conventional DNA Typewriter system, by setting the length of the spacer, which can bind to the remaining portions other than the target for the corresponding round in the DNA tape sequence, to be shorter than 16 bp.

Based on this, the present invention provides a composition for measuring or estimating elapsed time in cells, comprising:
(a) a prime editor protein or a nucleic acid encoding the same;
(b) a DNA tape comprising a disrupted monomer, which includes a disrupted protospacer adjacent motif (PAM), and active monomers, each including a PAM; and
(c) a prime editing guide RNA (pegRNA) which comprises a spacer and a reverse transcription template (RTT) and recognizes the DNA tape.

The present invention also provides isolated cells having introduced therein:
(a) a nucleic acid that encodes a prime editor so that the prime editor is expressed or is planned to be expressed;
(b) a DNA tape comprising a disrupted monomer, which includes a disrupted PAM, and active monomers, each including a PAM; and
(c) a prime editing guide RNA (pegRNA) which comprises a spacer and a reverse transcription template (RTT) and recognizes the DNA tape.

The prime editor is a type of gene editing system based on CRISPR/Cas9 derived from the bacterial immune system. Usually, wild-type Cas9 causes a DNA double-strand break in a specific sequence on the genome, which is complementary to the guide RNA. The prime editor protein is a genome editing method that can introduce genetic changes by breaking only one strand of DNA by fourth-generation gene editing technology without breaking both strands of DNA.

The prime editor may be, for example, one selected from the group consisting of PE1, PE2, PE3, and PE3b, PE4, PE5, PE6, and PE7, without being limited thereto.

The prime editor may be, for example, wild-type PE2, and specifically may be NGG-PAM-Cas9 nickase H840A comprising the nucleotide sequence of SEQ ID NO: 1.

The prime editor may comprise a variant that retains the same function. The variant may be a variant mutated to recognize NG-PAM.

The prime editor variant mutated to recognize NG-PAM may be one in which a specific nucleic acid sequence in the C-terminal region of the Cas9 nickase domain of wild-type PE2 is modified. For example, the prime editor variant mutated to recognize NG-PAM may contain at least one substitution selected from the group consisting of: substitution of nucleotides AG for CT at positions 3385 and 3386 in the nucleotide sequence of NGG-PAM-Cas9 nickase of SEQ ID NO: 1, which is wild-type; substitution of nucleotide T for A at position 3458 in the nucleotide sequence of NGG-PAM-Cas9 nickase of SEQ ID NO: 1; substitution of nucleotide A for G at position 3706 in the nucleotide sequence of NGG-PAM-Cas9 nickase of SEQ ID NO: 1; substitution of nucleotides ATTC for CGAA at positions 3708 to 3711 in the nucleotide sequence of NGG-PAM-Cas9 nickase of SEQ ID NO: 1; substitution of nucleotides AGG for GCC at positions 4018 to 4020 in the nucleotide sequence of NGG-PAM-Cas9 nickase of SEQ ID NO: 1; substitution of nucleotides GT for AG at positions 4057 and 4058 in the nucleotide sequence of NGG-PAM-Cas9 nickase of SEQ ID NO: 1; substitution of nucleotides GG for CC at positions 4064 and 4065 in the nucleotide sequence of NGG-PAM-Cas9 nickase of SEQ ID NO: 1; and substitution of nucleotide C for T at position 4155 in the nucleotide sequence of NGG-PAM-Cas9 nickase of SEQ ID NO: 1.

The prime editor comprises: (i) nickase or a dead editor; and (ii) Moloney murine leukaemia virus (M-MLV) reverse transcriptase (RT).

The nickase may be a variant of Cas protein modified to nick a single strand of DNA, and the dead editor may be a variant of Cas protein modified to bind to a target sequence but not induce DNA nicking.

The Cas protein may be Cas1, Cas1B, Cas2, Cas3, Cas4, Cas5, Cas6, Cas7, Cas8, Cas9, Cas10, Cas12a, Cas12b, Cas12c, Cas12d, Cas12e, Cas12g, Cas12h, Cas12i, Cas12j, Cas13a, Cas13b, Cas13c, Cas13d, Cas14, Csy1, Csy2, Csy3, Cse1, Cse2, Csc1, Csc2, Csa5, Csn2, CsMT2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3 or Csf4 endonuclease, without being limited thereto.

The Cas protein may be derived or isolated from a Cas protein ortholog-containing microorganism selected from the group consisting of Corynebacter, Sutterella, Legionella, Treponema, Filifactor, Eubacteriurn, Streptococcus (Streptococcus pyogenes), Lactobacillus, Mycoplasma, Bacteroides, Flaviivola, Flavobacteriurn, Azospirillurn, Gluconacetobacter, Neisseria, Roseburia, Parvibaculurn, Staphylococcus (Staphylococcus aureus), Nitratifractor, Corynebacterium, and Campylobacter. Alternatively, the Cas protein may be a recombinant protein.

The Cas protein may be Streptococcus pyogenes Cas9 (SpCas9). Recognition of a protospacer adjacent motif (PAM) recognition by SpCas9 is the critical first step of target DNA recognition, enabling SpCas9 to bind to and hydrolyze DNA. SpCas9, the most robust and widely used Cas9, primarily recognizes NGG PAMs.

The variant of Cas protein is a variant retaining the function of Cas nuclease, and examples thereof include, but are not limited to, xCas9, SpCas9-NG, Cas9 nickase (nCas9), dead Cas9 (dCas9), and destabilized Cas9 (DD-Cas9).

The nickase may be one in which at least one amino acid selected from the group consisting of D10, E762, H839, H840, N854, N863, and D986 of Cas9 is substituted with a different amino acid.

Streptococcus pyogenes Cas9 may contain a mutation in which at least one selected from the group consisting of catalytic aspartate residue at position 10 (D10), glutamic acid at position 762 (E762), histidine at position 840 (H840), asparagine at position 854 (N854), asparagine at position 863 (N863), and aspartic acid at position 986 (D986) is substituted with any different amino acid. Here, any different amino acid for substitution may be alanine, without being limited thereto.

In some embodiments, the Streptococcus pyogenes Cas9 protein may be mutated to recognize NGA (where N is any base selected from among A, T, G, and C), which is different from the PAM sequence (NGG) recognized by wild-type Cas9, by substituting at least one selected from among aspartic acid at position 1135 (D1135), arginine at position 1335 (R1335), and threonine at position 1337 (T1337), for example, all of the three amino acids, with a different amino acid.

For example, in the amino acid sequence of the Streptococcus pyogenes Cas9 protein, amino acid substitution may occur at
(1) D10, H840, or D10+H840;
(2) D1135, R1335, T1337, or D1135+R1335+T1337; or
(3) both residues (1) and (2).

The term “different amino acid” means an amino acid selected from among alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine, and all known variants of the amino acids thereof, exclusive of the amino acid found at the original mutation positions in the wild-type protein. In one example, the “different amino acid” may be alanine, valine, glutamine, or arginine.

The “reverse transcriptase (RT)” refers to an enzyme that uses RNA as a template and synthesizes new complementary DNA.
(a) The reverse transcriptase (RT) is an RNA-dependent DNA polymerase that can synthesize DNA strands (i.e., complementary DNA (cDNA)) using a reverse transcriptase template.
(b) Examples of the reverse transcriptase include, but are not limited to, Moloney murine leukemia virus (M-MLV) reverse transcriptase (RT) or a variant thereof, for example, RNase H activity-deficient M-MLV-RT or M-MLV RT variants (D200N, T306K, W313F, T330P, or L603W), bovine leukemia virus (BLV) RT or a variant thereof, Rous sarcoma virus (RSV) RT or a variant thereof, or avian myeloblastosis virus (AMV) RT or a variant thereof.

The prime editor and reverse transcriptase may individually comprise a prime editor protein and reverse transcriptase, respectively, or may be included in the form of a fusion protein of the prime editor protein and reverse transcriptase.

The prime editing guide RNA or DNA encoding the same comprises a binding site, which binds to the gene to be edited, and an editing sequence. The binding site may be arbitrarily located in the 5′ direction or 3′ direction of the reverse transcriptase template. Specifically, the binding site may be located in the 3′ direction of the reverse transcriptase template.

The binding site may comprise a sequence complementary to a genomic DNA strand nicked by a nuclease or variant thereof (e.g., nickase) contained in the prime editor protein. The binding site may hybridize to a target site, thereby serving as a target site for the initiation of reverse transcriptase activity.

The prime editing guide RNA comprises a guide sequence that recognizes the target sequence, a tracrRNA scaffold sequence, a primer binding site (PBS) required for the initiation of reverse transcription, and an RT template (RTT) that includes the desired genetic change.

The sequence comprising the editing sequence serves as a reverse transcriptase template. The reverse transcriptase template comprises the desired editing sequence and is homologous to the genomic DNA locus. The editing sequence is a heterologous sequence and comprises the target sequence to be edited in the genome.

The types of editing include substitution, insertion, deletion, etc., without being limited thereto. The types of editing include the type (e.g., A, G, C, T) or number (e.g., 1 bp, 2 bp, 3 bp, etc.) of nucleotides to be substituted, inserted, or deleted in the target sequence.

The editing position may be calculated based on the nick site. For example, the editing position may be expressed as +1, +2, +3, etc. from the nick site.

The “nick site” refers to the site in the target sequence, which is nicked by Cas9-nickase.

The “PAM” is a sequence essentially required for the Cas protein to bind to target DNA, and refers to the sequence located after the target nucleic acid. Bacteria store a portion of the sequence of an invading virus in a part of their genome, and this sequence is called the protospacer. Since the protospacer sequence is a partial sequence, the sequence adjacent thereto is the original sequence of the bacteria. The role of the PAM site is to prevent a sequence from being cleaved, unless the sequence is not flanked by the PAM, even though many sequences in bacteria may match the protospacer.

The “disrupted PAM” in the DNA tape sequence refers to a sequence that has been edited (substitution/insertion/deletion) by prime editing and can no longer function as the PAM.

A “monomer” is a repeating sequence unit present in the DNA tape, and in one monomer there is only one disrupted PAM or a PAM.

The “disrupted monomer” refers to a monomer having a disrupted PAM instead of a PAM, and the “active monomer” refers to a monomer having a PAM.

In the DNA tape sequence, two or more active monomers are repeatedly arranged in a row. In the DNA tape sequence, several active monomers are arranged in a line at the 3′ end of the disrupted monomer.

In the DNA tape sequence, several active monomers are arranged, and a disrupted monomer is present adjacent to the 5′ end of the active monomer that is present closest to the 5′ end among the active monomers. Here, the active monomer that is present closest to the 5′ end is hereinafter referred to as the “first active monomer”. In the 5′ direction of this disrupted monomer, the insert sequence and the disrupted monomer are alternately arranged. In this case, the spacer of the prime editing guide RNA is complementary to the 20-bp sequence in the 5′ direction of the PAM present in the first active monomer of the DNA tape. In the alternating arrangement of the disrupted monomers and the insert sequences, a 5′ portion exceeding 20 bp to which the space is to bind may be omitted.

When an environment in which prime editing can occur is created by treating cells with the composition, 1) a specific sequence is inserted at the 5′ side of the active monomer (hereinafter referred to as “insertion site”) in the target, and 2) the PAM in the first active monomer is disrupted. Likewise. successive sequence insertions as well as PAM disruption occur repeatedly in the 5′→3′ direction within the DNA tape. The specific process is as follows:

When the prime editing guide RNA recognizes a target including the PAM in the first active monomer and generates a DNA single-strand break (hereinafter referred to as “'nick”), a specific sequence inscribed in the RTT of the prime editing guide RNA is inserted between the first active monomer and simultaneous edit is installed within the PAM of the first active monomer, thereby depriving its potential to function as a PAM sequence. Accordingly, the first active monomer is changed to a disrupted monomer. The PAM in the active monomer at the next editing site located at the 3′ side of this monomer functions as a new PAM to be recognized by the prime editing guide RNA, and the target sequence of the prime editing guide RNA and the write-head shift in the 3′ direction of the DNA tape by one monomer unit. In the same manner, the prime editing guide RNA inserts a specific sequence into the next write-head, disrupts the PAM recognized in the corresponding round, and shifts the write-head in the 3′ direction again by one monomer unit. As this prime editing process is repeated, insertion of a specific sequence sequentially occurs between the monomers in the DNA tape in the 5′→3′ direction.

The DNA tape sequence is designed considering the following factors. The length of the disrupted PAM sequence in one disrupted monomer is referred to as i_{D}bp, and the length of the PAM sequence in one active monomer is referred to as i_{A}bp. The length of the sequence present at the 5′ side of the PAM or disrupted PAM in one monomer is denoted as j bp, the length of the sequence present in the 3′ direction with respect to the PAM or disrupted PAM sequence is denoted as k bp, and the length of the sequence inserted by prime editing is denoted as m bp. Accordingly, the length of one disrupted monomer is (j+i_{D}+k) bp, and the length of one active monomer is (j+i_{A}+k) bp. Here, i_{D}, i_{A}, j and k are integers greater than 0.

1) The spacer in the prime editing guide RNA should contain a k-bp sequence located at the 5′ side of the PAM, one or more disrupted monomers, and a portion or all of the insert sequence. That is, i_{D}, j and k are set to satisfy k+(j+i_{D}+k)<20. Additionally, as previously known, the spacer can bind to the target sequence and induce editing when it is 16 bp or longer in length. Thus, if the spacer is used with a reduced length, it is also possible to further limit the maximum value of k+(j+i_{D}+k) to less than 16.

2) The prime editing guide RNA can recognize and bind to a (j+k) bp sequence near the PAM of the active monomer in a portion other than the editing site for the corresponding round. In order to minimize monomer deletions caused by the prime editing guide RNA, (j+k) bp, which is the length of the sequence that binds to the target sequence, is set to be less than 16 bp. However, if the disrupted PAM sequence and the PAM sequence share the same sequence at their 3′ ends, the prime editing guide RNA can additionally bind to this sequence, and thus the length of (j+k) bp plus the length of the sequence identical between the PAM and the disrupted PAM is set to be less than 16 bp. For example, if the PAM sequence is TGG and the disrupted PAM sequence is TCG, the last ‘G’ of each of TG‘G’ and TC‘G’ are the same, and in this case, (j+k)+1<16 should be satisfied.

j represents an integer greater than 0, which is less than or equal to the distance between the PAM sequence in one monomer and the nick site induced by the prime editing guide RNA that binds to the 20-bp sequence adjacent to the 5′ side of the PAM.

i_{A} is determined by the PAM sequence to be used, and i_{A} is determined by the PAM sequence and the type of result of prime editing that disrupts the PAM sequence. If the result of prime editing inducing PAM disruption is substitution, i_{D}=i_{A}, if the result is insertion, i_{D}<i_{A}, and if the result is deletion, i_{D}<i_{A}.

For example, in the DNA tape, the disrupted monomer may comprise TCGNNNNNNNNNNN (where N is A, T, G, or C), and the active monomer may comprise TGGNNNNNNNNNNN (where N is A, T, G, or C).

The pegRNA comprises a spacer that recognizes a 20-bp sequence present at the 5′ side with respect to the PAM of the active monomer, a reverse transcription template (RTT) to be used for prime editing, and a primer binding site (PBS). The PBS is the same sequence as the 5′ portion with respect to the nick site of the target sequence, and the length thereof is variable, but the efficiency of prime editing may vary depending on the length of the PBS. The RTT is a portion that specifies the sequence of the result to be edited during prime editing, and the length thereof is also variable, but the efficiency of prime editing may vary depending on the length of the RTT. The RTT may comprise: 1) a specific sequence to be inserted; and 2) a portion or all of the disrupted monomer. However, if the RTT length exceeds the length of the insert sequence and the disrupted monomer, a repeating sequence of the active monomer may be included behind the disrupted monomer.

When the composition comprising the nucleic acid encoding the prime editor protein, the DNA tape, and the prime editing guide RNA is delivered into cells, the working time of prime editing in the cells may be measured and estimated by a method comprising steps of:
(a) transducing the composition into isolated cells and then culturing the cells;
(b) analyzing whether a sequence has been inserted into the DNA tape, and measuring the fraction of an inserted sequence at each insertion site of the DNA tape at time t, that is, the insertion frequency (IF_{i,t}) at the i^{th} insertion site at time t;
(c) measuring the frequency of the copy number of an intact sequence at each insertion site in the total copy number of the DNA tape sequence at time t, that is, the frequency of intact sequence (F_{i,t}) at the i^{th} insertion site at time t; and
(d) calculating the time elapsed from a given time point using the following equation:

$${F}_{i , t} = 1 - {IF}_{i , t} = {e}^{- {λ}_{i} ( t - {t}_{0} )} ( t \geq 0 , {t}_{0} \geq 0 )$$

wherein F_{i,t} represents the frequency (fraction) of the copy number of an intact sequence at the i^{th} insertion site relative to the total copy number of the DNA tape sequence, analyzed at any time point t, IF_{i,t} represents the frequency of an inserted sequence at the i^{th} insertion site, measured at any time point t, λᵢ is a positive constant that represents the rate of sequence insertion by prime editing at the i^{th} insertion site per unit time, and t₀ is the latent time taken for the composition transduced into the cells to be expressed.

In the present invention, step (b) of analyzing whether a sequence has been inserted into the DNA tape and measuring IF_{i,t} comprises obtaining a DNA sequence from cells that exhibit the prime editing activity of the transduced composition. This step of obtaining the DNA sequence may be performed using various DNA isolation methods known in the art.

Since it is considered that editing in the target sequence has occurred in each of the transduced cells, data may be obtained by performing sequencing of the target sequence, for example, deep sequencing or RNA sequencing.

In the present invention, step (c) comprises measuring F_{i,t} at each insertion site in the DNA tape at time t.

IF_{i,t} is calculated using the following equation (Park et al., Cell, 2021):

$${IF}_{i , t} = \frac{( Observed insertion frequency ) - ( background insertion frequency )}{100 - ( background insertion frequency )}$$

Then, in step (d), using the fact that F_{i,t} and IF_{i,t} satisfy the relationship F_{i,t}=1−IF_{i,t}, the time elapsed from a given time point in the cells is calculated using the following equation:

$${F}_{i , t} = 1 - {IF}_{i , t} = {e}^{- {λ}_{i} ( t - {t}_{0} )} ( t \geq 0 , {t}_{0} \geq 0 )$$

This time measurement method is based on the previous report that the frequency of intact target sequences decreases exponentially over time.

If the concentrations of the prime editor protein and prime editing guide RNA are kept constant, it is assumed that the sequence insertion reaction by prime editing is a first-order reaction based on the reaction rate law.

In the first-order reaction, the reaction rate is linearly proportional to the concentration of each reactant, and thus the rate of decrease in

$${F}_{i , t} ( \frac{{dF}_{i , t}}{{d}_{t}} )$$

may be expressed by the following equation:

$$\begin{matrix}( a ) & \\ \frac{{dF}_{i , t}}{{d}_{t}} = - {λ}_{i} {F}_{i , t} ( {λ}_{i} > 0 ) & equation ( 1 )\end{matrix}$$

The definite integral equation for time t in equation (1) above is as follows:

$$\begin{matrix}( a ) & \\ {F}_{i , t} = {e}^{- {λ}_{i} t} & equation ( 2 )\end{matrix}$$

As shown in equation (2) above, F_{i,t} follows the exponential decay that is used in radiometric dating.

The rate constant λᵢ may vary depending on the sequence composition of the target sequence when introducing the target sequence using lentiviral transduction, the concentrations of the prime editor and the prime editing guide RNA, and the type of cell line used for recording.

Herein, “editing” may be used interchangeably with “edit” or “edited”, and the term “gene editing technology” refers to a method of altering the nucleic acid sequence of a specific genomic target. Such a specific genomic target includes, but is not limited to, a chromosomal region, a gene, a promoter, an open reading frame, or any nucleic acid sequence.

As used herein, the term “target” or “target site” refers to a pre-identified nucleic acid sequence of any composition and/or length. Such a target includes, but is not limited to, a chromosomal region, a gene, a promoter, an open reading frame, or any nucleic acid sequence.

As used herein, “on-target” refers to a subsequence of a specific genomic target that may be completely complementary to a programmable DNA binding domain and/or a guide RNA sequence.

The terms “polypeptide”, “peptide” and “protein” are used interchangeably to refer to a polymer of amino acid residues, wherein the polymer may be conjugated to a moiety that does not consist of amino acids in the examples. The terms may apply to amino acid polymers in which one or more amino acid residues are artificial chemical mimetics of corresponding naturally occurring amino acids, as well as to naturally occurring amino acid polymers and non-naturally occurring amino acid polymers. A “fusion protein” may refer to a chimeric protein encoding two or more separate protein sequences that are recombinantly expressed as a single moiety.

The term “amino acid” may refer to naturally occurring and synthetic amino acids, as well as amino acid analogs and amino acid mimetics that function in a manner similar to the naturally occurring amino acids. Naturally occurring amino acids include those encoded by the genetic code, as well as those amino acids that are later modified, e.g., hydroxyproline, γ-carboxyglutamate, and O-phosphoserine. Amino acid analogs include compounds that have the same basic chemical structure as a naturally occurring amino acid, i.e., a carbon that is bonded to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but may retain the same basic chemical structure as a naturally occurring amino acid. Amino acid mimetics include chemical compounds that have a structure that is different from the general chemical structure of an amino acid, but that function in a manner similar to a naturally occurring amino acid. The terms “non-naturally occurring amino acids” and “non-natural amino acids” include amino acid analogs, synthetic amino acids and amino acid mimetics, which are not found in nature.

The invention is also directed to a nucleic acid encoding the composition.

The term “nucleic acid” is used interchangeably with “oligonucleotides”, “polynucleotides”, “nucleotides” and “nucleotide sequences”. The term may include a polymeric form of nucleotides of any length, either deoxyribonucleotides or ribonucleotides, or analogs thereof. Polynucleotides may have any three-dimensional structure, and may perform any function, known or unknown. A polynucleotide may comprise modified nucleotides, such as methylated nucleotides and nucleotide analogs. If present, modifications to the nucleotide structure may be imparted before or after assembly of the polymer.

The polynucleotide may typically comprise four nucleotide bases: adenine (A), cytosine (C), guanine (G), and thymine (T) (uracil (U) for thymine (T) when the polynucleotide is RNA).

The term “sequence” is the alphabetical representation of a molecule. This alphabetical representation may be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “nucleic acid” may include deoxyribonucleotides or ribonucleotides and polymers in either single-, double- or multiple stranded form, or complements thereof. The term “polynucleotide” may include a linear sequence of nucleotides.

The nucleic acid may be linear or branched. For example, the nucleic acid may be a linear chain of nucleotides, or the nucleic acid may be branched so that it makes up one or more nucleotide arms or branches.

The nucleic acid may be an RNA sequence, a DNA sequence, or a combination thereof (RNA-DNA combination sequence).

“Conservatively modified variations” may be applied to nucleic acid sequences. With respect to particular nucleic acid sequences, “conservatively modified variations” refers to those nucleic acids which encode identical or essentially identical amino acid sequences. Due to degeneracy of the genetic code, a large number of nucleic acid sequences can encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations”, which are one species of conservatively modified variations. Every nucleic acid sequence which encodes a polypeptide may also include every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule.

The term “isolated”, when applied to a nucleic acid or protein, denotes that the nucleic acid or protein is essentially free of other cellular components. Purity and homogeneity may be typically determined using analytical chemistry techniques such as polyacrylamide gel electrophoresis or high-performance liquid chromatography.

“Complementarity” or “complementary” refers to the ability of a nucleic acid to form hydrogen bond(s) with another nucleic acid sequence by either traditional Watson-Crick base pairing or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, and 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary”, as used herein, refers to a degree of complementarity that is at least 60%, 65%, 70%, 75%, 80%, 85%, 90%, or 95%. “Perfectly complementary” refers to a degree of complementarity that is at least 90%, 91%, 92%, 93%, 94%, 95%, 96%, 97%, 98%, 99% or 100% over a region of nucleotides, or refers to two nucleic acids that hybridize under stringent conditions.

The term “gene” means the segment of DNA involved in producing a protein and may include regions preceding and following the coding region (leader and trailer) as well as intervening sequences (introns) between individual coding segments (exons). The leader, the trailer and the introns include regulatory elements that are necessary during transcription and translation of a gene. Further, a “protein gene product” includes a protein expressed from a particular gene.

The nucleic acid may be delivered using a viral vector, for example, an adeno-associated viral vector (AAV), an adenoviral vector (AdV), lentiviral vector (LV) or a retroviral vector (RV), as well as other viral vectors such as episomal vectors containing Simian virus 40 (SV40) ori, bovine papilloma virus (BPV) ori, or Epstein-Barr nuclear antigen (EBV) ori, as well as virus-like particles (VLPs) or engineered virus-like particles (eVLPs).

The vector may be delivered in vivo or into cells through microinjection (e.g., direct injection into a lesion or target site), electroporation, lipofection, viral vector, nanoparticles, protein translocation domain (PTD) fusion proteins, etc.

For delivery, a known expression vector such as a plasmid vector, a cosmid vector, or a bacteriophage vector may be used, and the vector may be easily produced by those skilled in the art according to any known method using DNA recombination technology. The vector may be a viral vector or a plasmid vector, and the viral vector may specifically be a lentiviral vector or a retroviral vector. However, the present invention is not limited thereto, and those skilled in the art can freely use known vectors as long as the purpose of the present invention can be achieved.

For viral vectors, virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell.

In certain cases, vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Moreover, certain vectors are capable of directing the expression of genes to which they are operatively linked. Such vectors are referred to herein as “expression vectors”. Common expression vectors of utility in recombinant DNA techniques are often in the form of plasmids.

Recombinant expression vectors may comprise a nucleic acid in a form suitable for expression of the nucleic acid in a host cell, which means that the recombinant expression vectors comprise one or more regulatory elements, which may be selected on the basis of the host cells to be used for expression, that is operatively-linked to the nucleic acid sequence to be expressed.

Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” may include promoters, enhancers, internal ribosomal entry sites (IRES), and other expression regulatory elements (e.g., transcription termination signals, such as polyadenylation signals and poly-U sequences). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific.

In some embodiments, a vector may comprise one or more pol III promoters, one or more pol II promoters, one or more pol I promoters, or combinations thereof. Examples of pol III promoters include, but are not limited to, U6 and HI promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter.

The term “regulatory elements” may include enhancer elements, such as WPRE; CMV enhancers; and the intron sequence between exons 2 and 3 of rabbit β-globin. It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including encoded by nucleic acids as described herein (e.g., clustered regularly interspaced short palindromic repeat (CRISPR) transcripts, proteins, enzymes, mutants thereof, fusion proteins thereof, etc.). Advantageous vectors include lentiviruses and adeno-associated viruses, and types of such vectors can also be selected for targeting particular types of cells.

Vectors may contain one or more marker sequences suitable for use in the identification and/or selection of cells which have or have not been transformed or genomically modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., β-galactosidase, alkaline phosphatase or luciferase), and genes which visibly affect the phenotype of transformed or transfected cells, hosts, colonies, or plaques. Any vector suitable for the transformation of a host cell, (e.g., E. coli, mammalian cells such as CHO cell, insect cells, etc.) as embraced by the present invention, for example vectors belonging to the pUC series, pGEM series, pET series, pBAD series, pTET series, or pGEX series. In some embodiments, the vector is suitable for transforming a host cell for recombinant protein production. Methods for selecting and engineering vectors and host cells for expressing gRNAs and/or proteins (e.g., those provided herein), transforming cells, and expressing/purifying recombinant proteins are well known in the art.

Examples of the cells include, but are not limited to, eukaryotic cells (e.g., embryonic cells, stem cells, somatic cells, germ cells, etc.) derived from fungi such as yeast, eukaryotic animals, and/or eukaryotic plants, cells derived from eukaryotic animals (e.g., primates such as humans, monkeys, dogs, pigs, cattle, sheep, goats, mice, rats, etc.), or cells derived from eukaryotic plants (e.g. algae such as green algae, corn, soybeans, wheat, rice, etc.).

The DNA sequence encoding the prime editor protein, the DNA tape, and the DNA sequence encoding the prime editing guide RNA may be provided through a delivery means such as a vector. The DNA sequence encoding the prime editor protein, the DNA tape, and the DNA sequence encoding the prime editing guide RNA may be placed on the same vector, so that they may be delivered simultaneously by the single vector. The DNA sequence encoding the prime editor protein, the DNA tape, and the DNA sequence encoding the prime editing guide RNA may be placed on different vectors and delivered by the vectors.

In some embodiments, the sequence encoding the prime editor protein and the prime editing guide RNA may be delivered in mRNA form. The mRNA may be delivered directly into cells or delivered by a carrier.

Furthermore, an RNP (ribonucleoprotein) complex formed by assembling the prime editor protein (1) and the mRNA of the prime editing guide RNA (2) may be delivered. The RNP may be delivered directly or delivered by a carrier.

The RNP complex may be delivered into cells by various methods known in the art, such as microinjection, electroporation, DEAE-dextran treatment, lipofection, nanoparticle-mediated transfection, protein transduction domain-mediated introduction, and PEG-mediated transfection, without being limited thereto.

The carrier may comprise, for example, a cell-penetrating peptide (CPP), nanoparticles, or a polymer, without being limited thereto. CPPs are short peptides that facilitate cellular uptake of a variety of molecular cargoes (from nanosized particles to small chemical molecules and large fragments of DNA). With respect to the nanoparticles, the composition according to the present invention may be delivered by polymer nanoparticles, metal nanoparticles, metal/inorganic nanoparticles, or lipid nanoparticles.

### EXAMPLES

Hereinafter, the present invention will be described in more detail with reference to examples. These examples are only for illustrating the present invention, and it will be apparent to those of ordinary skill in the art that the scope of the present invention is not to be construed as being limited by these examples.

### Example 1. Establishment of PE2max Knock-In Cell Line Expressing Prime Editor

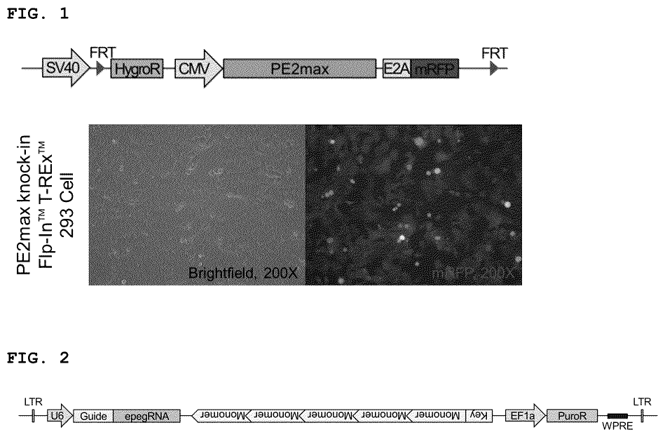

A monoclonal cell line expressing PE2max and hygromycin-resistance genes and mRFP fluorescence was produced using the Invitrogen™ Flp-In™ System and used in subsequent experiments (FIG. 1). FIG. 1 shows a vector construct used to produce PE2max knock-in cells and the expression of mRFP fluorescence by the produced PE2max knock-in cells.

### Example 2. Novel DNA Typewriter System

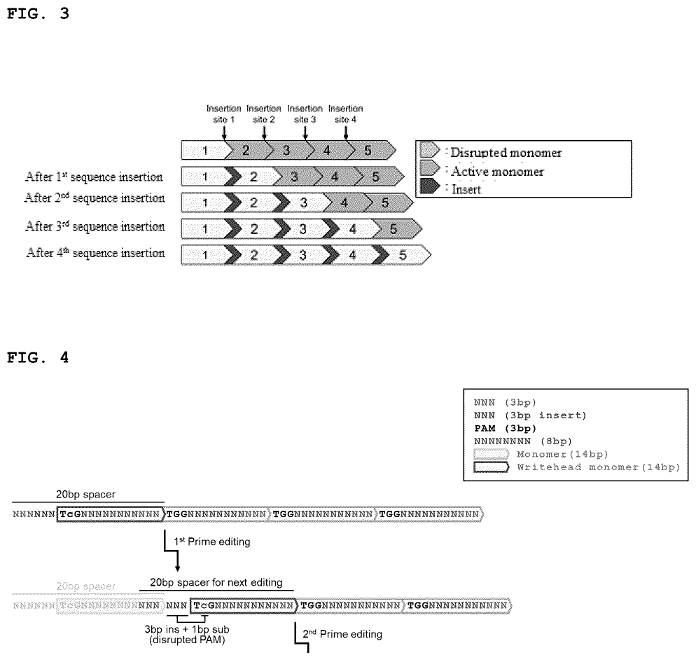

A lentiviral vector was constructed by cloning a DNA tape, which comprises a target sequence, and a prime editing guide RNA that can cause 3-bp sequence insertion and 1-bp substitution in the DNA tape (FIG. 2). The constructed vector was delivered into PE2max knock-in cells at a multiplicity of infection (MOI) of 0.3 so that one DNA tape and one prime editing guide RNA were introduced per cell. A schematic representation of the DNA tape is shown in FIG. 3. When editing occurs repeatedly as shown in FIG. 3, the active monomers included in the write-heads are sequentially changed to disrupted monomers, and a specific sequence is inserted between the monomers. The DNA tape has a structure in which a 3-bp sequence (GAG) at the 3′ end of the monomer, a 3-bp insert sequence (GTA), and a 14-bp disrupted monomer (TCGCCGGAGCTGAG; SEQ ID NO: 2) are arranged side by side, and then four active monomers (TGGCCGGAGCTGAG; SEQ ID NO: 3) are arranged in a tandem array (FIG. 3).

(1) Primer editing guide RNA:
(SEQ ID NO: 4)
GAGGTATCGCCGGAGCTGAGGTTTTAGAGCTAGAAATAGCAAGTTAAAAT
AAGGCTAGTCCGTTATCAACTTGAAAAAGTGGCACCGAGTCGGTGCTCAG
CTCCGGCGATACCTCAGCTCCGGCGCGCGGTTCTATCTAGTTACGCGTTA
AACCAACTAGAATTTTTTT
(2) DNA tape:
(SEQ ID NO: 5)
GAGGTATCGCCGGAGCTGAGTGGCCGGAGCTGAGTGGCCGGAGCTGAGTG
GCCGGAGCTGAGTGGCCGGAGCTGAG

If the five monomers of the DNA tape are referred to as monomers 1 to 5 according to the numbers assigned to the monomers in FIG. 3, the working principle of the novel DNA Typewriter is as follows (FIG. 4).

In the first round of editing, a sequence consisting of a combination of the 3-bp sequence located in front of monomer 1 (the same sequence as the last 3 bases of the monomer; GAG), the 3-bp insert sequence (GTA), and the 14-bp disrupted monomer (monomer 1) acts as a site to which a 20-bp spacer binds, and the first prime editing occurs. Insertion of a 3-bp nucleotide sequence at the 1^{st} insertion site occurs and at the same time, the PAM (TGG) of monomer 2 is changed to TCG by G-to-C substitution, causing PAM disruption, and thus monomer 2 is converted from an active monomer to a disrupted monomer. In the same manner, a combination of the 3-bp sequence at the 3′ end of monomer 1, the 3-bp insert sequence inserted in the first round of editing, and monomer 2 acts as a site to which the 20-bp spacer binds, and second prime editing occurs. At this time, insertion of a 3-bp sequence at the 2^{nd} insertion site occurs and at the same time, the PAM of monomer 3 is disrupted, and a combination of the 3-bp sequence at the 3′ end of monomer No. 2, the 3-bp insert sequence inserted in the second round of editing, and monomer 3 acts as a new site to which the 20-bp spacer binds. As such events occur repeatedly, 3-bp sequence insertions at insertion sites 1 to 4 occur sequentially.

After the DNA tape and the prime editing guide RNA were transduced into PE2max knock-in cells by lentivirus, the cells were harvested at intervals of 2 to 4 days while the cells were maintained for a predetermined period of time (28 days), and the DNA tape sequence was extracted from the genomic DNA (gDNA) of the cells. The DNA tape sequence was analyzed by NGS to check whether the intended sequence was inserted at each insertion site.

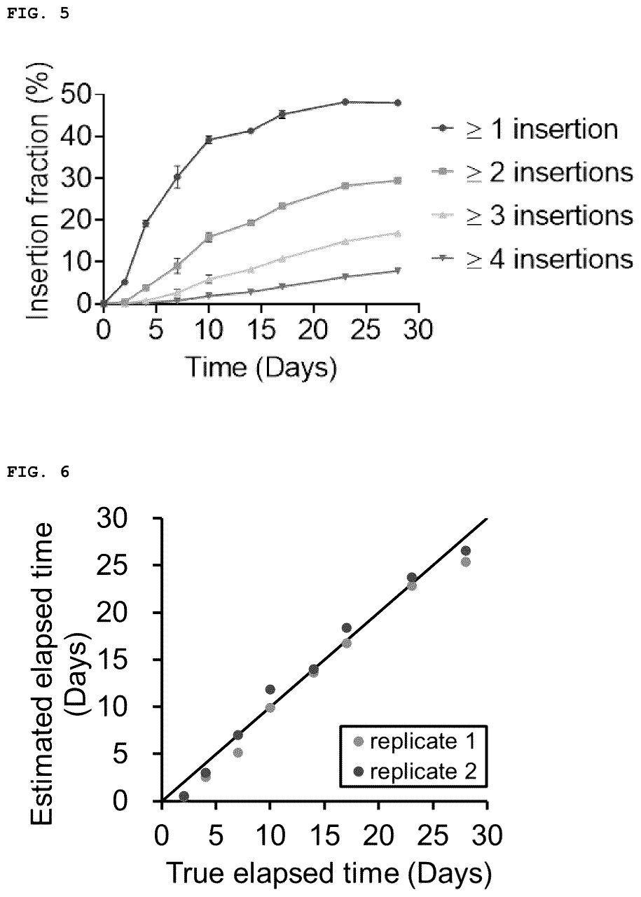

FIG. 5 shows the fraction of reads with insertion among all reads analyzed (%) depending on the number of sequences inserted into the DNA tape sequence at each measurement time point. Referring to FIG. 5, it can be confirmed that the number of sequence insertions increased over time.

### Example 3. Checking of Elapsed Time

F_{i,t} was calculated for each insertion site depending on elapsed time and was fitted with an exponential decay curve for each insertion site. Table 1 below shows the results of calculating the relative absolute error (RAE) and the mean RAE between the estimated elapsed time and the true elapsed time in each of two independent replicates using the decay curves.

TABLE 1

| True | replicate 1 | | replicate 2 | |
|---|---|---|---|---|
| elapsed | estimated | | estimated | |
| time | elapsed time | RAE (%) | elapsed time | RAE (%) |
| 2 | 0.46 | 76.79 | 0.52 | 73.88 |
| 4 | 2.53 | 36.70 | 3.00 | 25.10 |
| 7 | 5.21 | 25.63 | 7.04 | 0.64 |
| 10 | 9.93 | 0.72 | 11.87 | 18.74 |
| 14 | 13.66 | 2.44 | 14.02 | 0.17 |
| 17 | 16.78 | 1.30 | 18.43 | 8.43 |
| 23 | 22.90 | 0.43 | 23.77 | 3.35 |
| 28 | 25.37 | 9.41 | 26.61 | 4.95 |
| | mean RAE (%) | 19.18 | mean RAE (%) | 16.91 |

FIG. 6 shows the results of comparing the estimated elapsed time and the true elapsed time based on the exponential decay curves fitted to the experimental data shown in Table 1.

### Example 4. Monomer Deletion Events in Conventional DNA Typewriter

Regarding DNA Typewriter, Choi et al., Nature, Vol. 608, Aug. 4, 2022 describes a DNA Typewriter system that continuously install insertions into a DNA tape sequence using one or multiple types of pegRNA.

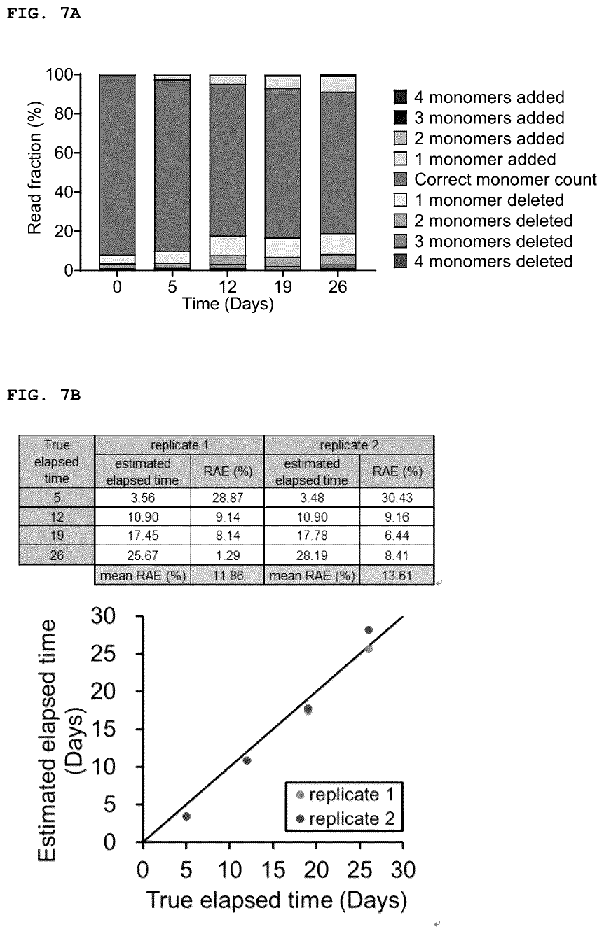

An experiment was conducted using the TAPE-1 sequence provided in Choi et al., Nature, Vol. 608, Aug. 4, 2022, and monomer deletion events (FIG. 7A), estimated elapsed time, and mean RAE values (FIG. 7B), which occurred as a result of the experiment, were checked.

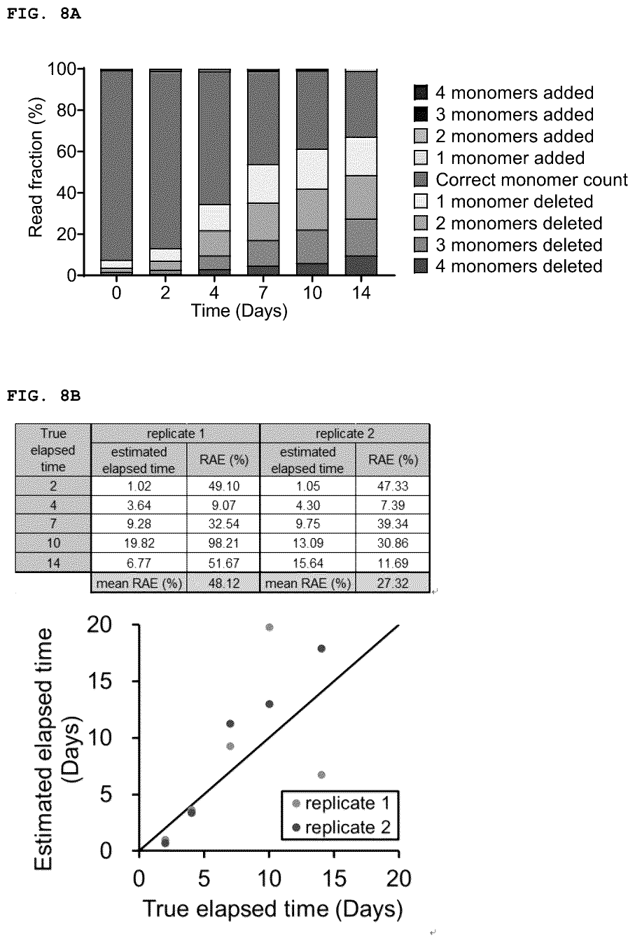

As a result of conducting the experiment using the DNA Typewriter sequence predicted to be the best for prime editing among all possible DNA Typewriter constructs, notable monomer deletion events occurred incrementally as time passed (FIG. 8A).

The estimated elapsed time results and the mean RAE values (FIG. 8B) were checked. The DeepPrime algorithm (Volume 186, Issue 10, 11 May 2023, Pages 2256-2272.e23) was used to predict prime editing efficiency for sequence selection.

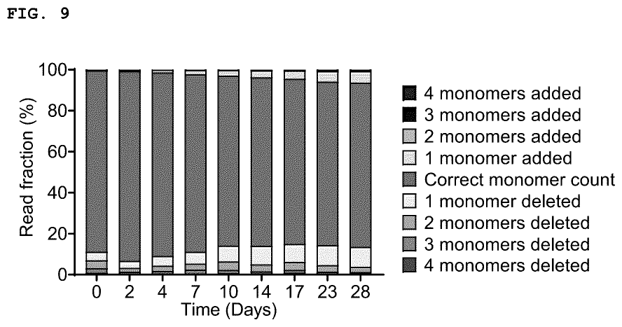

On the other hand, as a result of checking the number of monomers observed for 28 days when using the DNA clock construct according to the present invention (Example 2), it was confirmed that monomer deletions did not tend to increase compared to those in the DNA Typewriter construct (FIG. 9).

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

What is claimed is:

1. A composition for measuring or estimating elapsed time in isolated cells, comprising:
(a) a prime editor protein or a nucleic acid encoding the same;
(b) a DNA tape comprising a disrupted monomer, which includes a disrupted protospacer adjacent motif (PAM), and active monomers, each including a PAM; and
(c) a prime editing guide RNA (pegRNA) which comprises a spacer and a reverse transcription template (RTT) and recognizes the DNA tape.

2. The composition according to claim 1, wherein the active monomers are arranged in a row at a 3′ end of the disrupted monomer.

3. The composition according to claim 2, wherein a specific base sequence is inserted into a write-head by prime editing, and the PAM in the active monomer at an editing site is disrupted.

4. The composition according to claim 1, wherein each disrupted monomer in the DNA tape has a length of (j+i_{D}+k) bp, and each active monomer in the DNA tape has a length of (j+i_{A}+k) bp,
wherein
i_{D}, i_{A}, j and k are integers greater than 0,
the disrupted PAM has a length of i_{D} bp,
the PAM has a length of i_{A}bp,
a sequence present in the 5′ direction with respect to the PAM or the disrupted PAM has a length of j bp;
a sequence present in the 5′ direction with respect to the PAM or the disrupted PAM has a length of k bp; and
a sequence inserted by prime editing has a length of m bp.

5. The composition according to claim 4, wherein the prime editing guide RNA that recognizes the DNA tape comprises a target-binding site of (j+k) bp (j+k<16).

6. The composition according to claim 1, wherein the prime editor comprises: (i) nickase or a dead editor; and (ii) Moloney murine leukaemia virus (M-MLV) reverse transcriptase (RT).

7. The composition according to claim 6, wherein the nickase is one in which at least one amino acid selected from the group consisting of D10, E762, H839, H840, N854, N863, and D986 of Cas9 is substituted with a different amino acid.

8. The composition according to claim 7, wherein the different amino acid is alanine.

9. Isolated cells having introduced therein:
(a) a nucleic acid that encodes a prime editor so that the prime editor is expressed or is planned to be expressed;
(b) a DNA tape comprising a disrupted monomer, which comprises a disrupted PAM, and active monomers, each comprising a PAM; and
(c) a prime editing guide RNA (pegRNA) which comprises a spacer and a reverse transcription template (RTT) and recognizes the DNA tape.

10. A method for measuring or estimating elapsed time in isolated cells, comprising steps of:
culturing the isolated cells of claim 9;
harvesting the cultured cells at any time point t, and then analyzing whether a sequence has been inserted into the DNA tape from a genomic DNA of the cells;
based on analyzing whether the sequence has been inserted into the DNA tape, measuring a fraction of an inserted sequence at each insertion site in copies of the DNA tape sequence at time t, that is, an insertion frequency (IF_{i,t}) at an i^{th} insertion site at time t;
measuring a frequency of a copy number of an intact sequence at each insertion site in a total copy number of the DNA tape sequence at time t, that is, a frequency of intact sequence (F_{i,t}) at the i^{th} insertion site at time t; and
calculating a time elapsed from a given time point using the following equation:
$${F}_{i , t} = 1 - {IF}_{i , t} = {e}^{- {λ}_{i} ( t - {t}_{0} )} ( t \geq 0 , {t}_{0} \geq 0 )$$
wherein F_{i,t} represents the frequency (fraction) of the copy number of an intact sequence at the i^{th} insertion site relative to the total copy number of the DNA tape, analyzed at any time point t, IF_{i,t} represents the frequency of an inserted sequence at the i^{th} insertion site, measured at any time point t, λᵢ is a positive constant that represents the rate of sequence insertion by prime editing at the i^{th} insertion site per unit time, and t₀ is the latent time taken for the composition transduced into the cells to be expressed.
